(19) United States
(12) Patent Application Publication
(10) Pub. No.: US 2013/0231695 A1
(43) Pub. Date: Sep. 5, 2013

(54) EMBOLIC COIL

(71) Applicant: TUFTS MEDICAL CENTER, INC., Boston, MA (US)

(72) Inventor: Adel M. Malek, Lexington, MA (US)

(73) Assignee: Tufts Medical Center, Inc., Boston, MA (US)

(21) Appl. No.: 13/782,182

(22) Filed: Mar. 1, 2013

Related U.S. Application Data
(60) Provisional application No. 61/605,313, filed on Mar. 1, 2012.

(51) Int. Cl. A61B 17/12

(52) U.S. Cl. CPC A61B 17/1215
USPC 606/200

(57) ABSTRACT

An apparatus includes an embolic coil including a mechanically flexible tubular member having a central lumen and a plurality of expandable sheaths spaced along the axial length of the tubular member, each expandable sheath enveloping and corresponding to the plurality of apertures, the expandable sheaths configured to have an initial low profile arrangement and upon introduction of an expansion medium to the central lumen of the tubular member radially expands to a high profile arrangement. Methods for making and using the apparatus are also described.

### CROSS-REFERENCE TO RELATED APPLICATIONS

This application claims the benefit of U.S. Provisional Application No. 61/605,313 filed Mar. 1, 2012. The contents of U.S. Provisional Application No. 61/605,313 are incorporated herein in its entirety.

### BACKGROUND

This invention relates to coils, such as embolic coils, as well as related methods, devices and compositions.

A brain aneurysm, also called an intracranial aneurysm, is an abnormal bulge or ballooning in a blood vessel supplying the brain. The weakened area forms a sac that fills with blood. Intracranial aneurysms can rupture and cause bleeding into the brain. Usually this occurs in the area between the brain and the surrounding membrane (the arachnoid), called the subarachnoid space, causing a subarachnoid hemorrhage. Subarachnoid hemorrhage resulting from a ruptured intracranial aneurysm occurs approximately 35,000 times per year in the United States.

Currently, intracranial aneurysms are treated by microsurgical clipping or endovascular coiling. In the latter, the goal is to prevent aneurysm rupture by inserting a thin wire into the aneurysm forming a coiled structure which blocks blood flow into the aneurysm. In some treatment paradigms, intracranial stents are used within the blood vessel to buttress placement of coils

### SUMMARY

In a general aspect of the invention, an apparatus comprising an embolic coil including a mechanically flexible tubular member having a central lumen; and a plurality of expandable sheaths spaced along the axial length of the tubular member, each expandable sheath configured to have an initial low profile arrangement and upon introduction or withdrawal of an expansion medium to the central lumen of the tubular member radially expands to a high profile arrangement.

Embodiments of this aspect of the invention may include one or more of the following features. The tubular member includes apertures, spaced along the axial length of the tubular member, each aperture corresponding to one of the expandable sheaths and each configured for introduction or withdrawal of the expansion medium. Each of the expandable sheaths includes an inflatable balloon.

The expandable medium can be an embolizing agent in a variety of forms (e.g., liquid gas). For example, the embolizing agent can comprise a biocompatible polymer, prepolymer, polyethelene glycol, derivatives of polyethelene glycol, or a hydrogel. Other embolizing agents can include Onyx®, Neucrylate AN™ or other non-adhesive liquid embolic agents typically used for the pre-surgical embolization of intracranial brain Arteriovenous malformations (bAVM).

The apparatus includes a microcatheter configured to deliver the embolic coil to a treatment site, the microcatheter having a guidewire and a release mechanism configured to detachably release the embolic coil from a distal end of the microcatheter.

In another aspect of the invention, a method of making an embolic coil includes providing a mechanically flexible tubular member having a central lumen; providing a plurality of expandable sheaths along the axial length of the tubular member, each expandable sheath configured to have an initial low profile arrangement and upon introduction or withdrawal of an expansion medium to the central lumen of the tubular member radially expands to a high profile arrangement.

Embodiments of this aspect of the invention may include one or more of the following features. The method further includes providing apertures, spaced along the axial length of the tubular member, each aperture corresponding to one of the expandable sheaths and each configured for introduction or withdrawal of the expansion medium. The method further includes forming each of the expandable sheaths as an inflatable balloon. The method further includes providing an embolizing agent comprising one or more of a biocompatible polymer, prepolymer, polyethelene glycol, derivatives of polyethelene glycol, a hydrogel, Onyx®, Neucrylate AN™.

In still another aspect of the invention, a method of treating a patient includes affixing, to a distal end of a microcatheter, an embolic coil including a mechanically flexible tubular member having a central lumen and a plurality of expandable sheaths spaced along the axial length of the tubular member; delivering the microcatheter with the embolic coil through the vasculature of the patient to a treatment site; and providing an expansion medium to at least one of the plurality of expandable sheaths to expand the at least one sheath from an initial low profile arrangement to a high profile arrangement.

Among other advantages, an embolic coil having expandable sheaths allows for complete occlusion and embolism of an aneurysm. Once the embolic coil is positioned within the aneurysm thrombosis is enhanced. Introducing the embolic coil within the aneurysm using a delivery system (e.g., catheter) is made easier because the coil is introduced in a low profile arrangement. Once properly positioned with the aneurysm, the expandable sheaths of the embolic coil are expanded into a higher profile arrangement. In this higher profile arrangement, blood flow in the aneurysm is blocked by virtue of the expandable sheaths filling the volume within the aneurysm. Furthermore, in their expanded, higher profile condition, the embolic coil is contained.

Furthermore, the extent to which the expandable sections of the embolic coil are expanded to fill the volume of the aneurysm can be controlled by the surgeon or operator. For example, the surgeon or operator can position the embolic coil and then fill the expandable sheaths such that the coil loops establish a foothold within the aneurysm. In this way, once the embolic coil is detached from the delivery catheter, the risk of the embolic coil are portions of the coil being released from the aneurysm outside the neck of the aneurysm and into the vasculature is minimized, which otherwise might lead to distal embolism and stroke with undesirable clinical effects.

Modes for carrying out the present invention are explained below by reference to an embodiment of the present invention shown in the attached drawings. The above-mentioned object, other objects, characteristics and advantages of the present invention will become apparent from the detailed description of the embodiment of the invention presented below in conjunction with the attached drawings.

### DESCRIPTION OF DRAWINGS

FIG. 1 is a schematic side sectional view representation of an embolization delivery system including a microcatheter;

FIG. 2 is a side view of a portion of an embolic coil shown in its unfurled state and configured to be detachably connected to the distal end of the microcatheter of FIG. 1;

FIG. 3 is the embolic coil of FIG. 2 disposed within an aneurysm and in its unexpanded state; and

FIG. 4 is the embolic coil of FIG. 2 disposed within an aneurysm and in its expanded state.

FIG. 5 is an alternative embodiment of an embolic device.

### DESCRIPTION

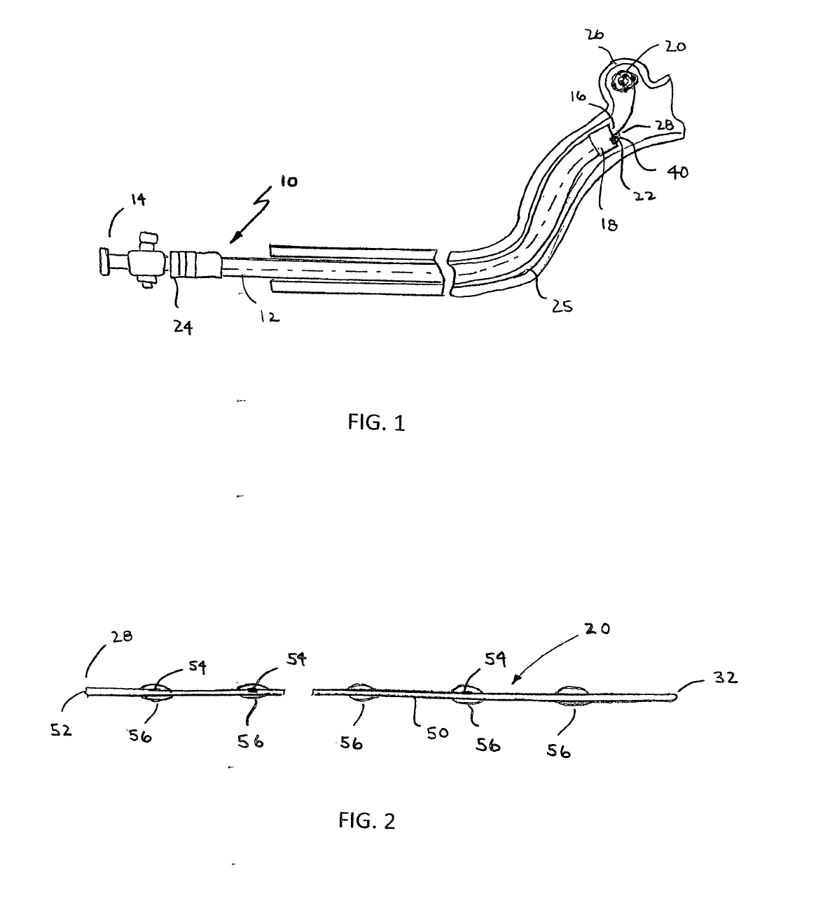

Referring to FIG. 1, an embolization delivery system 10 includes a microcatheter 12 having a proximal end 14 and a distal end 16, a connector 18, a detachable embolic coil 20, and a release mechanism 22. The proximal end 14 of the microcatheter 12 may be coupled to or pass through a manifold 24 for use with procedures that include a microcatheter 12 or other delivery mechanisms. In general, embolization delivery system 10 establishes a pathway through the vasculature 25 of a patient. In this embodiment, microcatheter 12, here coupled to manifold 24, is first inserted into the vasculature 25 of the patient to a preselected or targeted location. In this particular application, the microcatheter 12 is delivered to an aneurysm 26. The distal end 16 of the microcatheter 12 is capable of being inserted into the vasculature 25 of the patient and positioned proximate to an aneurysm 26 or other abnormality in the vasculature 25. In other embodiments, embolization delivery system 10 includes a stainless-steel, nitinol, or other metallic guidewire for facilitating delivery of the embolic coil 20 to the aneurysm 26.

Connector 18 has a proximal portion that is disposed around and permanently coupled to the distal end 16 of the microcatheter 12. A proximal end 28 of embolic coil 20 is disposed within the connector 18 and securely held in place by compressive forces. In addition, the embolic coil 20 is substantially linear as it progresses through the microcatheter 12 due to the boundary constraints placed upon the coil 20 by the microcatheter 12. However, upon exiting the distal end 16 of the microcatheter 12, the distal end 32 of the embolic coil 20 will curl or coil in a pre-determined shape previously effected during the design and manufacturing stage order to occlude the flow of fluid to the aneurysm 26 or other abnormality in the vasculature 25.

In one embodiment, the microcatheter 12 includes a release mechanism 40 for detaching the embolic coil 20 once it is positioned with the aneurysm 26. For example, the release mechanism can include a wire 42 that is manipulated by a physician or other attendant. Manipulating the wire 42 reduces or eliminates the compressive forces exerted by the connector 18 onto the proximal end 28 of the embolic coil 20, thereby, allowing the coil to detach from the microcatheter 12. For example, manipulation of wire 42 by the surgeon can involve pulling or moving the wire 42 in a manner that causes the coil 20 to be released into the vasculature, in particular, the aneurysm 26.

Alternative detachment or release mechanisms may also be used to detach the embolic coil from the distal end of the microcatheter. For example, an electrolytic, a mechanical ball-socket arrangement or through the use of a local electrically generated heating of a polymeric attachment zone are suitable means of releasing the embolic coil.

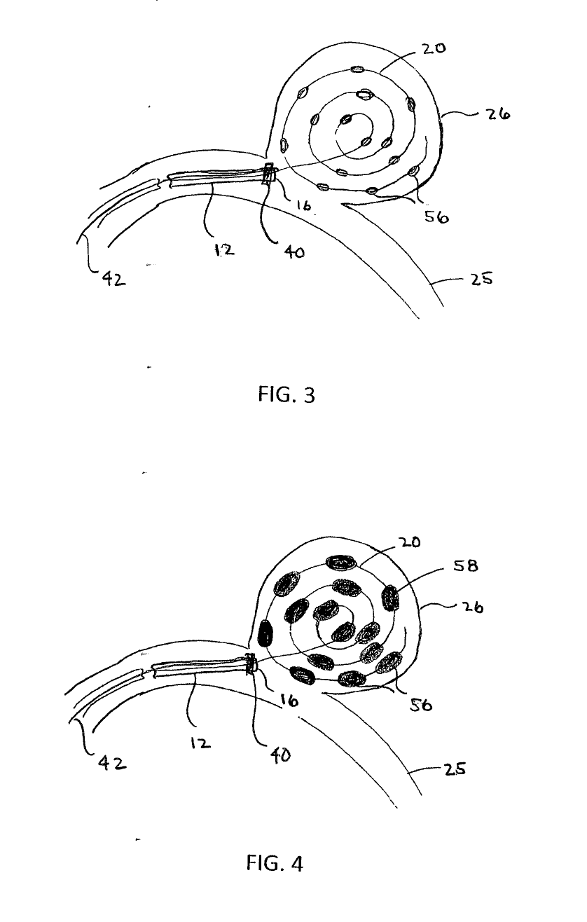

Referring to FIG. 2, embolic coil 20 is shown in its spread and unfurled state. Embolic coil 20 includes a flexible cylindrical member 50 having a central lumen 52 and a series of spaced-apart apertures 54 along its length. Each aperture 54 is enclosed by an expandable sheath 56 which envelopes the cylindrical member 50 around the aperture.

Embolic coil 20 is formed of a metal filament such as platinum, platinum alloy (e.g., platinum-tungsten alloy), stainless steel, or shape-memory alloys (e.g., Nitionol). In certain embodiments, embolic coil 20 can be formed of one or more polymers, such as polyolefins, polyurethanes, block copolymers, polyethers, and polyimides, The expandable sheaths are formed of, for example, an elastomeric material which may comprise, but is not limited to, polymeric materials, latex, rubber, silicon rubber, Pebax®, urethane, pelothane, Tecothane®, polyester isobutyl styrene, epoxies and thermoplastics

It is important to appreciate that the size and spacing of apertures 54 as well as the associated expandable sheaths 56 may be non uniform or irregular. Furthermore, the stiffness of the membrane of the expandable sheaths may be non-uniform with each unit or between units

In certain embodiments, a catalyst or an accelerant may be coated on the balloon at different concentrations to affect the reaction. Alternatively the surface coating of the balloon may be conductive and a current could be passed through the microcatheter 12 so as to start a catalyst process or to induce a swelling reaction of a local hydrogel or affect the rate of a chemical reaction needed for solidification of the liquid polymer, hydrogel or other liquid embolic material. The current to the balloon coating surface could be provided by another embedded wire or conductor within the delivery microcatheter or guidewire.

Referring to FIG. 3, in operation, microcatheter 12 is guided, for example, via a guidewire to the aneurysm 26. Once the microcatheter 12 is positioned near the neck of the aneurysm 26, embolic coil 20 is extended within the aneurysm. As the embolic coil 20 is pushed from the distal end 16 of microcatheter 12 into the aneurysm 26, the distal end 32 of the embolic coil begins to curl or coil thereby filling the volume of the aneurysm. Even at this point, embolic coil 20 can initiate a clotting or thrombotic reaction within the aneurysm that can decrease bleeding from the aneurysm. In certain procedures a stent may be passed first into the parent artery to serve as a scaffold for the coils (“stent-assisted coiling”), for example, as discussed in WO 2012/102919, which is incorporated herein by reference.

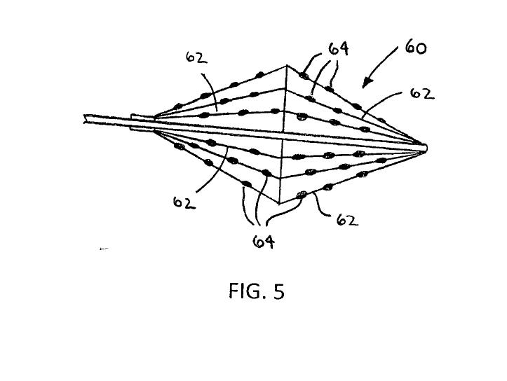

Referring to FIG. 4, to further fill the volume of and embolize the aneurysm 26, an embolizing agent 58 is introduced through microcatheter 12, through central lumen 52 and into expandable sheaths 56. As the embolizing agent is introduced, each expandable sheath 56 swells to fill the volume within the aneurysm 26. The embolizing agent generally has sufficient viscosity for being retained in the expandable sheaths 56.

Embolizing agent can be in a variety of forms and can include a number of different compositions. For example, the embolizing agent can be in the form of a liquid embolizing agent such as Onyx®, a product of ev3 Endovascular, Inc., Plymouth, Minn. The liquid embolizing agent can also include a non-adhesive liquid embolic agent such as Neucrylate AN™, typically used for the pre-surgical embolization of intracranial brain Arteriovenous malformations (bAVM). Other suitable liquid embolizing agents can include biocompatible polymers, prepolymers, or polyethelenes (e.g., polyethelene glycol). The embolizing agent may also be 1-Hexyl n-cyanoacrylate compound (Neucrylate™ AN) or include a hydrogel.

Once the expandable sheaths 56 are filled or inflated, the surgeon can use the release mechanism 40 to release the embolic coil and then withdraw the microcatheter 12 from the vasculature 25. Because the expandable sheaths 56 are expanded after being introduced into the aneurysm the risk of the embolic coil 20 or portions of the coil being released into the vasculature 25 are virtually eliminated. Furthermore, the embolic coil 20 with its expandable sheaths 56 expanded serves a scaffolding framework that is controllable by the surgeon.

It is important to appreciate that once the embolizing agent 58 is introduced within the expandable sheaths 56, the surgeon can withdraw all or some of the embolizing agent 58 using suction to reduce the degree to which the expandable sheaths 56 are filled. For example, the surgeon may wish to withdraw a portion of the embolizing agent, reposition the emboli coil 20 and then re-expand the expandable sheaths 56.

As discussed above, a variety of liquid embolic agents having different viscosities can be used. Moreover, after introducing a liquid embolic agent to the expandable sections, a different agent serving as a catalyst can be introduced to cause the liquid embolic agent to change characteristics (e.g., harden).

The structure described above in conjunction with FIGS. 1-4 was also linear in nature. Other embodiments of the embolic coil may have arrangements that when released into the aneurysm is 3-dimensional or stent-like.

Referring to FIG. 5, for example, an embolic coil 60 has an umbrella-like structure with each hollow rib or vane 62 of the umbrella structure including spaced parts expandable sheaths 64, each sheath corresponding to an aperture (not shown) on the rib.

Although the structure described above in conjunction with FIGS. 1-4 are in the form of a single channel lumen, in other embodiments, embolic coil 20 can include other structures such as multiple channel structures that may be nested. In such embodiments, the multiple channels provide an initial structure that is multi-dimensional and may be advantageous for larger aneurysms. Furthermore, in such situations multiple embolic coils can be introduced, either together or sequentially, into the aneurysm and are individually controlled.

The structure described above in conjunction with FIGS. 1-4 was also linear in nature. Other embodiments of the embolic coil may have arrangements that when released into the aneurysm is 3-dimensional or stent-like. For example, in one embodiment, the embolic coil has an umbrella-like structure with each rib or vane of the umbrella structure including spaced parts expandable sheaths. Thus, the term “coil” is not intended to be restricted to linear single path structures but is intended to encompass a wide variety of geometries suitable for filling the aneurysm.

It is to be understood that the foregoing description is intended to illustrate and not to limit the scope of the invention, which is defined by the scope of the appended claims. Other embodiments are within the scope of the following claims. For example, other structures for flexible catheters used to deliver detachable/releasable embolic coils, as well as the structures for the embolic coils themselves are encompassed by the claims. The variety of delivery systems (e.g., catheters)and coils described in U.S. Provisional Application No. 61/605,313 are also encompassed by the claims. For example, FIG. 8 of U.S. Provisional Application No. 61/605,313 and the accompanying description in the specification shows an embolic coil having expandable sheaths delivered by a microcatheter to an aneurysm. The description of silk-based as well as other non-silk compositions suitable for use as an embolizing agent U.S. Provisional Application No. 61/605,313 are also encompassed by the claims.

What is claimed is:

1. An apparatus comprising:
an embolic coil including:
a mechanically flexible tubular member having a central lumen; and
a plurality of expandable sheaths spaced along the axial length of the tubular member, each expandable sheath configured to have an initial low profile arrangement and upon introduction or withdrawal of an expansion medium to the central lumen of the tubular member radially expands to a high profile arrangement.

2. The apparatus of claim 1 wherein the tubular member includes a plurality of apertures, spaced along the axial length of the tubular member, each aperture corresponding to one of the plurality of expandable sheaths and each configured for introduction or withdrawal of the expansion medium.

3. The apparatus of claim 1 wherein each of the plurality of expandable sheaths includes an inflatable balloon.

4. The apparatus of claim 1 wherein the expandable medium is a liquid embolizing agent.

5. The apparatus of claim 4 wherein the liquid embolizing agent comprises a biocompatible polymer or prepolymer.

6. The apparatus of claim 5 wherein the liquid embolizing agent comprises a polyethelene glycol.

7. The apparatus of claim 6 wherein the liquid embolizing agent comprises derivatives of polyethelene glycol.

8. The apparatus of claim 6 wherein the liquid embolizing agent comprises a hydrogel.

9. The apparatus of claim 1 further comprising a microcatheter configured to deliver the embolic coil to a treatment site, the microcatheter including a guidewire.

10. The apparatus of claim 9 wherein the microcatheter includes a release mechanism configured to detachably release the embolic coil from a distal end of the microcatheter.

11. A method of making an embolic coil comprising:
providing a mechanically flexible tubular member having a central lumen;
providing a plurality of expandable sheaths along the axial length of the tubular member, each expandable sheath configured to have an initial low profile arrangement and upon introduction or withdrawal of an expansion medium to the central lumen of the tubular member radially expands to a high profile arrangement.

12. The method of claim 11 further comprising providing a plurality of apertures, spaced along the axial length of the tubular member, each aperture corresponding to one of the plurality of expandable sheaths and each configured for introduction or withdrawal of the expansion medium.

13. The method of claim 11 further comprising forming each of the plurality of expandable sheaths as an inflatable balloon.

14. The method of claim 11 further comprising providing the expandable medium as a liquid embolizing agent.

15. The method of claim 14 further comprising providing the liquid embolizing agent as a biocompatible polymer or prepolymer.

16. The method of claim 15 further comprising providing the liquid embolizing agent as a polyethelene glycolor a derivative of polyethelene glycol.

17. The method of claim 14 further comprising providing the liquid embolizing agent as a hydrogel.

18. The method of claim 11 further comprising providing a microcatheter configured to deliver the embolic coil to a treatment site, the microcatheter including a guidewire.

19. The method of claim 18 further comprising providing a release mechanism configured to detachably release the embolic coil from a distal end of the microcatheter.

20. A method of treating a patient comprising:
affixing, to a distal end of a microcatheter, an embolic coil including a mechanically flexible tubular member having a central lumen and a plurality of expandable sheaths spaced along the axial length of the tubular member;
delivering the microcatheter with the embolic coil through the vasculature of the patient to a treatment site;
providing an expansion medium to at least one of the plurality of expandable sheaths to expand the at least one sheath from an initial low profile arrangement to a high profile arrangement.
